(19) United States
(12) Patent Application Publication
(10) Pub. No.: US 2017/0065187 A1
(43) Pub. Date: Mar. 9, 2017

(54) HEART RATE PLAYBACK MACHINE

(71) Applicants: Jackson Hsieh, City of Industry, CA (US); Alexander Keerthi Mylavarapu, City of Industry, CA (US)

(72) Inventors: Jackson Hsieh, City of Industry, CA (US); Alexander Keerthi Mylavarapu, City of Industry, CA (US)

(73) Assignee: PARADIGM HEALTH AND WELLNESS, City of Industry, CA (US)

(21) Appl. No.: 14/847,472

(22) Filed: Sep. 8, 2015

(51) Int. Cl. A61B 5/0255; A63B 22/00; A63B 23/12; A63B 22/06; A63B 24/00; A63B 21/06; A61B 5/00; A63B 22/02

(52) U.S. Cl. CPC A61B 5/0255; A61B 5/486; A61B 5/0022; A61B 5/0017; A63B 22/0076; A63B 22/025; A63B 22/0605; A63B 22/0664; A63B 24/0087; A63B 21/06; A63B 23/1218; A63B 23/1227; A63B 23/1236; A63B 2024/0093

(57) ABSTRACT

Improvements in a heart rate playback machine to initially monitor heart rate of a person performs cardiovascular exercise. The heart rate is generally monitored by a chest strap that is placed around the chest of a user that transmits either the beats or the calculated heart rate. The heart rate can be received by a monitoring device, like a wrist or waist mounted device. The heart rate is recorded while the person is exercising. The machine then convert the heart rate into estimated loads of resistance, speed, elevation or other changes to simulate duplication of the heart rate. The machine uses a reverse algorithm to increase the resistance, speed or elevation prior to the recorded heart rate changes. The recorded heart rate, or the result of the algorithm, is then transportable or playable on the piece of exercise equipment. The exercise can be scaled to accommodate athletes of a different age or fitness level.

### CROSS REFERENCE TO RELATED APPLICATIONS

This application claims the benefit of Provisional Application Ser. No. 62/120,478 filed Feb. 25, 2015 the entire contents of which is hereby expressly incorporated by reference herein.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

Field of the Invention

This invention relates to improvements in heart rate playback machine. More particularly, the present heart rate playback machine records a person's heart rate changes as they exercise and then converts the recorded heart rate changes into intensity changes in a fitness machine to duplicate the heart rate changes to make an equivalent workout.

Description of Related Art Including Information Disclosed Under 37 CFR 1.97 and 1.98.

Many people monitor their heart rate to maintain workout intensity to optimize the cardiovascular benefits of the exercise. Initially heart rate was monitored by manually sensing heart beats over a period of time. More modern technology utilize a chest strap, wrist strap or finger sensor that monitors heart beats and then displays the present heart rate. Some systems will store a maximum reading or an average heart rate while a person exercises. Some pieces of exercise equipment have integrated heart rate monitors that display heart rate, and still other pieces of fitness equipment can also alter the intensity of the workout by changing the load, speed or elevation on the fitness equipment to alter the heart rate of a person.

A number of patents and or publications have been made to address these issues. Exemplary examples of patents and or publication that try to address this/these problem(s) are identified and discussed below.

U.S. Pat. No. 8,033,959 issued Oct. 11, 2011 to Mark Arthur Oleson discloses a Portable fitness monitoring systems, and applications thereof. The portable fitness monitoring system includes a portable fitness monitoring device for sensing performance parameters during a physical activity conducted by the user and communicating performance parameter data to the dedicated portable fitness monitoring device. While the device monitors heart rate and performance of a user it does not record and track the heart rate changes over an extended period of time where the heart rate can be used to create an equivalent future workout.

U.S. Pat. No. 8,152,695 issued on Apr. 10, 2012 to Raymond W. Riley et al., disclose an Athletic performance sensing and/or tracking systems and methods. The athletic performance sensing and/or tracking systems include components for measuring or sensing athletic performance data and/or for storing and/or displaying desired information associated with the athletic performance to the user (or others). While the system senses and tracks performance of an athlete, the accumulated information is tracked as opposed to continuously recorded to enable the system to recreate the workout.

U.S. Pat. No. 8,911,330 issued on Dec. 16, 2014 to Scott R. Watterson et al., disclose an Indoor-Outdoor Exercise System. The remote communication system is receptive to the data regarding the outdoor exercise routine and generates an exercise program based on the data regarding the outdoor exercise routine. The device uses real-time information to generate an exercise routine. It does not use historical collected heart rate information to produce the heart rate stress on indoor exercise equipment. It further does not allow the heart rate changes to be transported to other pieces of equipment.

U.S. Patent Application Publication 2009/0023554 was published on Jan. 22, 2009, discloses an Exercise System in Virtual Environment. The exercise system includes at least two exercise modules and is arranged to allow multiple users performing exercises on, with or against the modules in different locations while performing at least one preset task defined in a context of a story, a scenery or a video (or computer) game each in turn preferably defined in a virtual environment. While this system provides a virtual environment for multiple users to compete, the exercise environments are pre-created from stories or games and a user is not able to enter a workout based upon their own heart rate changes as they exercise.

What is needed is a monitoring system that tracks and records heart rate over the exercise period. The recorded date and then be interpolated to recreate the workout on exercise equipment. The proposed heart rate playback machine provides the solution with a monitoring and recording system combined with re-creation algorithm.

### BRIEF SUMMARY OF THE INVENTION

It is an object of the heart rate playback machine to monitor changes in the heart rate of a user as a person exercises. The heart rate is generally monitored by a chest strap that is placed around the chest of a user that transmits either the beats or the calculated heart rate. The heart rate can be received by a monitoring device, like a wrist or waist mounted device. The chest belt can syncs directly to an exercise machine wirelessly to transfer a previous workout or to a smart device and the smart device can connect to the machine, or the smart device can upload the data to the internet.

It is another object of the heart rate playback machine to record the heart rate as a person exercises. There are several ways to record the heart rate changes, including but not limited to a time between heartbeats, the actual heart rate over at particular time intervals or a period of time that a heart rate stays at different heart beats per minute. The total time interval of the workout is also recorded.

It is another object of the heart rate playback machine to convert the heart rate into estimated loads of resistance, speed, elevation or other changes to simulate duplication of the heart rate. The machine uses a reverse algorithm to increase the resistance, speed or elevation prior to the recorded heart rate changes. The reverse algorithm can use each workout to better tune when the intensity is adjusted to try and match when a present heart rate changes are made to correspond to a prior recorded heart rate.

It is another object of the heart rate playback machine to be able to send or transport the historical workout heart rate or the simulated workout to another. This allows a person to bring the workout with them, or can send the work out to a friend that can enjoy the workout.

It is still another object of the heart rate playback machine to playback the exercise. The intensity can also be scaled to accommodate an athlete that may have higher or lower peak heart rate. This can allows older or younger athletes to replay the workout. When the replay is on a treadmill it is possible to select all of the changers to the treadmill as speed only, elevation only or a combination thereof.

It is still another object of the heart rate playback machine to utilize the chest belt to automatically count every time the user performs an activity, when a certain “stress” is put on the heart. This chest belt is sensitive enough to determine if a user is standing or sitting. If a user does 10 squat exercises, bench presses, pull-ups, swim strokes, etc. . . . the chest belt will detect 10 “stresses” put on the heart and can count the reps and display it on a computer, tablet, phone, watch or other smart device.

Various objects, features, aspects, and advantages of the present invention will become more apparent from the following detailed description of preferred embodiments of the invention, along with the accompanying drawings in which like numerals represent like components.

### BRIEF DESCRIPTION OF THE SEVERAL VIEWS OF THE DRAWING(S)

FIG. 1 shows a flow chart of the chain of events associated with the heart rate playback machine.

FIG. 2 shows a pictorial view of communication between the devices.

FIG. 3 shows a person on a bench press with the weight against the chest of the person.

FIG. 4 shows a person on a bench press with the weight being lifted thereby stressing the person.

FIG. 5 shows heartbeat graph.

### DETAILED DESCRIPTION OF THE INVENTION

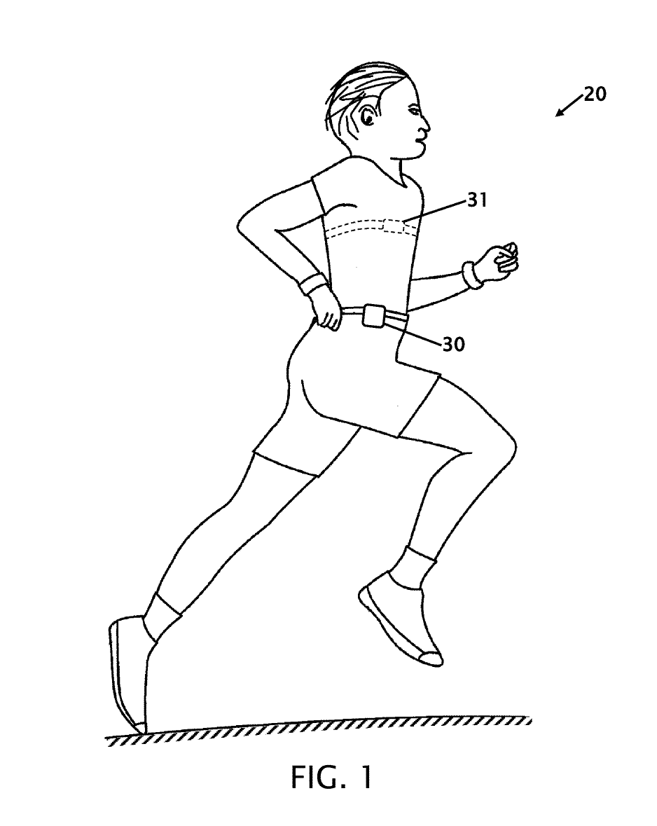

FIG. 1 shows the chain of events associated with the heart rate playback machine. To create a workout a first user 20 uses a heart rate monitor 31 that communicates with the receiver 30. The first user 20 can select a start button on the receiver 30 or the receiver can continuously monitor the heart rate and when a sufficiently prolonged and increasing heart rate is detected the receiver can estimate the point in time when the first user 20 began to exercise. The receiver 30 can also use GPS, motion sensors or gyros to determine that the first user has begun an exercise workout.

The heart rate monitor is typically a chest belt, but could also be other types of heart rate monitor 20 can also have an integrated receiver, but in either case, the heart rate information is recorded and the user's IBI (inter-beat interval) and/or BPM (Beats Per Minute) while the user is outside performing cardiovascular activities. (Running, biking, swimming, tennis, boxing, dancing etc. . . . ). It is also contemplated that the heart rate information for the workout can be recorded in different ways, including but not limited to the actual heart rate over at particular time intervals or a period of time that a heart rate stays at different heart rate beats per minute.

After the exercise from the first user 20 is recorded, the first user 20 can then download the recorded information to a computer of piece of exercise machine. The download can be a wired connection, infrared, or a wireless connection such as an inductive coupling, Wi-Fi, Bluetooth, RF, infrared or other wireless communication. The heart rate can be received by a monitoring device, like a wrist or waist mounted device. The chest belt can syncs directly to an exercise machine wirelessly to transfer a previous workout or to a smart device and the smart device can connect to the machine, or the smart device can upload the data to the internet.

The exercise equipment 40 can then essentially re-create a pseudo equivalent exercise experience by altering the speed and/elevation changes to provide similar heart beat changes that were experienced with outdoor training. Given that there is a lag time from when a load or exercise intensity changes until the heart rate change is reasonably altered the exercise equipment 40 can optionally interpolate the heart rate at a period in time of between 10 to 30 seconds later in the recorded workout to get a heart rate of the second user 21 at the same heart rate at approximately the same time as the first user 20.

After the information from the first user has been downloaded and stored into the exercise machine to create the exercise workout. The exercise machine can then alter the load by speed, resistance and or elevation to give essentially the same cardio workout as the outdoor/non-machine activity.

As an example, if the first user 20 runs 5 miles on a mountain trail, the heart rate data from the first user 20 is recorded the whole time and the first user can come back to their treadmill 40 and program the treadmill to increase/decrease speed+elevation to match (in a cardiovascular BPM sense) the outdoor run.

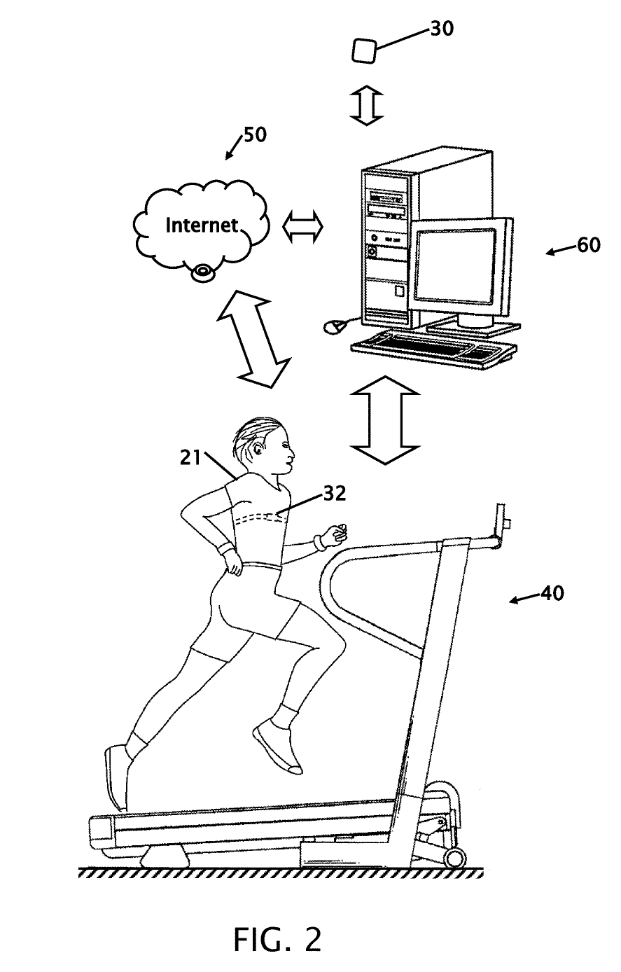

FIG. 2 shows a pictorial view of communication between the devices. In this figure the recording device 30 transmits data to either a computer 60, into the internet 50 or to the fitness device 40. When the recorded data is available on the internet a user can transfer the information to another person, to a storage device like a flash drive or to their database on the computer or to their gym. The computer 60 can be used to scale the data for other users or for other types of fitness equipment like a bicycle or stepping machine.

Option A

After the above data has been recorded and transmitted to a treadmill or other type of exercise equipment. The workout can be stored for use by other people 21 or recalled for future exercise. The exercise machine 40 will remember the actual speed/resistance/incline parameters that resulted in the same cardio workout and saved into the machine computer. The speed and elevation changes can be separated from the heart rate. This would allow a user to identify the workout as a “5 mile run, mile 1 at a pace of 7 min/mi pace, mile 2 at a pace of 7.5 min/mile pace, etc. . . . ” on the machine. Without entering the speed and elevation changed the speed/elevation/resistance changes would be based purely on the heart rate data that was saved. The fitness machine 40 would always be increasing the speed/resistance as the user increases in athletic ability to allow the athlete to get into better physical condition.

As an example when a first user 20 runs a particular route for the first time, the runner establishes a “personal best” for the run. As the runner continues to run the same route their athletic ability will increase that the run will become easier. If the treadmill maintains the same pace, without input from the heart rate the runner would not improve their ability or fitness level. When the machine 40 uses the heart rate of the user as one parameter to increase the speed and/or elevation the ability of the athlete will improve. While the exercise time for the runner can decrease as the runner gets faster, the exercise equipment can extend portions of the run or can loop the one or more portions of the run/workout to maintain the same duration of the run, if desired by the runner.

Option B

The computer, microprocessor in the exercise machine or a smart device will be able to send both the physical parameters of the computer, microprocessor in the exercise machine and the user's 21 heart beats per minute is ported to another piece of exercise machine by a (wirelessly connection). When the first user returns from an exercise routing, the first user 20 interfaces with the exercise equipment 40, the internet 50 or the computer 60, the heart rate from the workout is downloaded and synced.

Once the information is in the computer 60 or the internet 50, the data from the workout can be converted or sent to a friend or to an application that can be sold, purchased or given to other such as friends or team mates. This allows other 21 to experience the exact same run or workout as the first user 20. The data transfer can include treadmill parameters of “speed/incline”. If the second athlete 21 is at a different level of fitness, the workout can be adjusted based upon miles, pace or incline, the data and heart rate is transferred to the second athletes 21 exercise equipment. The other treadmill, bicycle or stepper makes adjustments so the second athlete 21 experiences an essentially equivalent cardiovascular workout.

Option C

In this third preferred embodiment, the workout is transferrable between different types of exercise equipment where the changes in exercise intensity is usable for a variety of purposes. As an example, if a first athlete 20 prefers exercise by running outside, but the second person has bad knees and they can exercise by cycling, the heart rate changes can be transferred between different types of exercise equipment at the same time or at different times. The beats-per-minute or inter-beat interval data can be transferred to any machine in order to mirror a previous workout.

All of the heart rate information can be encoded and uploaded onto the web or a web site such as mycloudfitness.com so first 20 or the second/subsequent 21 user can track their heart rate variability, beats per minute information. The heart rate information can be transferred to a smart device like a phone, computer, tablet, watch, flash drive or music player. The data could then be used for comparison by both the first user 20, the second user 21 or by a third parts such as a coach or trainer. This chest belt or other heart rate monitoring device could also compare different activities that don't require a fitness machine 40. The other activities can include, but not be limited to swimming vs. shooting under pressure.

The exercise equipment monitors the first athlete and/or a subsequent athlete to compare the heart rate of first athlete and/or a subsequent athlete using said down loaded logged historical heart rate information to alter the speed, elevation or resistance to improve time overlap of real-time heartrate with the loaded logged historical heart rate information.

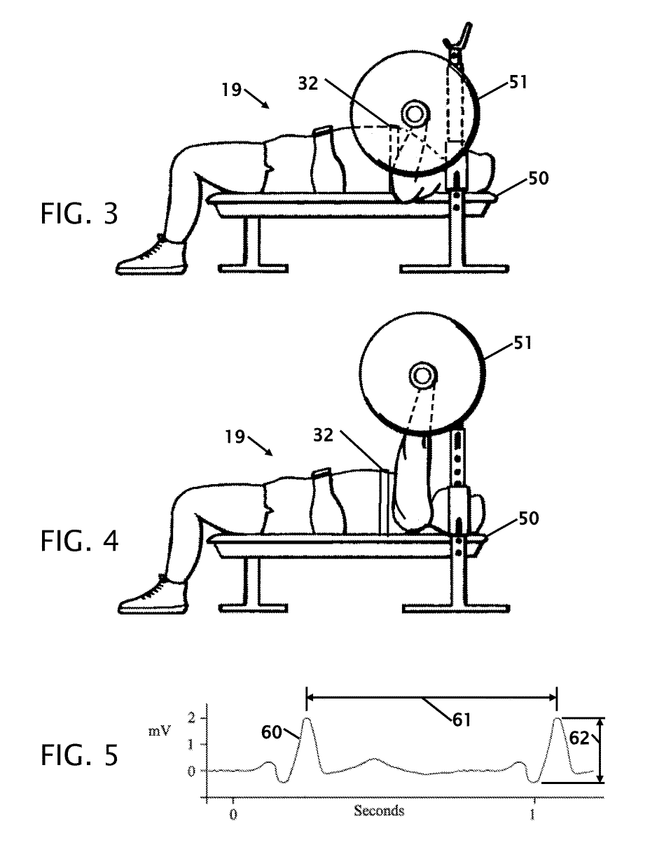

FIG. 3 shows a person 19 on a bench press bench 50 with the weight 51 against the chest of the person 19, and FIG. 4 shows a person 19 on a bench press bench 50 with the weight 51 being lifted thereby stressing the person 19 that is detectable with the chest strap monitor 32.

In another contemplated embodiment the chest belt is sensitive enough to determine when the heart is stressed. FIG. 5 shows heartbeat graph. In the heartbeat graph, each heartbeat 60 has certain period between beats 61 and each beat has a particular height 62 that approximately equates to the pressure wave or volume of blood being pumped. When a person is at a steady state of exercise the period 61 and the height 62 of the heart beat is fairly consistent. When a person exhorts a lift, such as shown in the bench press, the period 61 is altered and/or the height 62 of the beat is altered depending upon when the lift begins and ends relative to when a heartbeat occurs. The variation in the period and/or the height of the beat is used to determine that a lift or stress has occurred.

The chest belt 32 can use the stress to automatically count every time the user performs an activity, when a certain “stress” is put on the heart. This chest belt 32 is also sensitive enough to determine if a user is standing or sitting. If a user 19 does a bench press, squat exercises, pull-ups, swim strokes, etc. . . . the chest belt will detect change in heartbeat period and/or beat height as “stresses” put on the heart and can count the reps and display it on a computer, tablet, phone or other smart device. The smart device can record and use the counts in an application that includes features like a weight selection, an exercise type, a coaching module and a movie.

Thus, specific embodiments of a heart rate playback machine have been disclosed. It should be apparent, however, to those skilled in the art that many more modifications besides those described are possible without departing from the inventive concepts herein. The inventive subject matter, therefore, is not to be restricted except in the spirit of the appended claims.

1. A heart rate playback machine comprising:
a first athlete using a heart rate monitor while said first athlete performs an athletic workout;
said heart rate monitor logs historical heart rate information over a period of said athletic workout;
down loading said logged historical heart rate information, and
using said down loaded logged historical heart rate information at a future time or date to create a workout on a piece of exercise equipment based upon said logged historical heart rate information.

2. The heart rate playback machine according to claim 1 wherein said piece of exercise equipment is selected from a group consisting of a treadmill, a stationary bicycle, rowing machine or a stepping machine.

3. The heart rate playback machine according to claim 2 wherein said exercise equipment change speed, elevation or resistance to re-create said logged historical heart rate information for said first athlete and/or a subsequent athlete.

4. The heart rate playback machine according to claim 3 wherein said exercise equipment is the same type of exercise equipment or a different type of exercise equipment.

5. The heart rate playback machine according to claim 3 wherein said exercise equipment monitors said first athlete and/or a subsequent athlete to compare the heart rate of first athlete and/or a subsequent athlete using said down loaded logged historical heart rate information to alter said speed, elevation or resistance to improve time overlap of real-time heartrate with said loaded logged historical heart rate information.

6. The heart rate playback machine according to claim 1 wherein said logged historical heart rate is transferrable using a wireless connection.

7. The heart rate playback machine according to claim 4 wherein said wireless connection is selected from a group of connections consisting of inductive coupling, Wi-Fi and Bluetooth.

8. The heart rate playback machine according to claim 1 wherein said logged historical heart rate is scalable based upon a physical fitness level of a subsequent athlete.

9. The heart rate playback machine according to claim 1 wherein said logged historical heart rate is transferrable over an internet connection or with removable memory device.

10. The heart rate playback machine according to claim 1 wherein said heart rate information is transferred to a smart device consisting of a group of a phone, tablet or music player

11.-20. (canceled)
